(19) United States
(12) Patent Application Publication
(10) Pub. No.: US 2024/0427953 A1
(43) Pub. Date: Dec. 26, 2024

(54) METHOD AND SYSTEMS FOR SELECTING A HEATING ARRANGEMENT

(71) Applicant: LMK Thermosafe Limited, Haverhill, Suffolk (GB)

(72) Inventors: Vahaj Mohaghegh, Haverhill, Suffolk (GB); Hassan Shirvani, Haverhill, Suffolk (GB); Javaid Butt, Haverhill, Suffolk (GB); Mark Newton, Haverhill, Suffolk (GB); Jamie Evans, Haverhill, Suffolk (GB)

(73) Assignee: LMK Thermosafe Limited, Haverhill, Suffolk (GB)

(21) Appl. No.: 18/690,233

(22) PCT Filed: Sep. 7, 2022
(86) PCT No.: PCT/EP2022/074833

§ 371 Date: Mar. 7, 2024

(30) Foreign Application Priority Data
Sep. 8, 2021 (GB) 2112810.3

(51) Int. Cl. G06F 30/17

(52) U.S. Cl. CPC G06F 30/17

(57) ABSTRACT

Disclosed herein is a method for calculating a time to establish a phase change of a material in a predetermined container arrangement subject to a heating arrangement. The method comprises the steps of: determining a set of basis functions from a plurality of temperature field snapshots for a corresponding material: representing an interface between a solid region of the material and a liquid region of the material as a boundary surface: iteratively updating the boundary surface over time based on calculated heat flux across the boundary to thereby update a volume of the solid region and a volume of the liquid region for the material in the container arrangement, each iteration comprising, calculating for the liquid region, using a temperature dependent thermal diffusivity function, a temperature distribution for the liquid region as a combination of the basis functions of the set of basis functions; and determining a time to establish a phase change of the material contained in the predetermined arrangement based on a convergence condition for the iterative updating.

### FIELD OF THE INVENTION

The present invention relates to methods for selecting a heating arrangement, such as for liberating solid cargo from containers in liquid form. In particular methods of determining a time to establish a phase change in a material held in a container arrangement subject to a heating arrangement are provided.

### BACKGROUND

Every year, large quantities of cargo involving materials having melting points from around room temperature up to around a few hundred degrees Celsius are packaged and transported both domestically and internationally, often by road, sea and even air transportation. To allow for effective handling of such materials during transportation and storage, the cargo is usually packaged into containers which are sealed and more easily moved. The containers can take on a variety of forms, but may be, for example, a drum, a barrel, or a so-called intermediate bulk container (IBC), which is a standardised, reusable, multi-use industrial-grade container, typically constructed out of polymer and/or metal. The cargo material is typically loaded into containers whilst in a liquid form, however once in the container the material may often solidify due to the ambient temperature and pressure that the container is subject to.

Of course it may be necessary at points in the cargo's journey to discharge the material from the container. For example to transfer to a further container or a parcel tanker for part of the journey, or to a shore based storage facility whilst the cargo awaits onward transportation, such as by road. Equally, when the cargo reaches its final destination it is necessary to discharge the material from the container in which it resides. For material in a liquid state this can be easily achieved such as by pumping (or pouring) the material from the container. However, should the material have solidified in transit then, in order to liberate (or discharge), the material from the container it may be necessary to melt the material to transition the material back to its liquid phase.

Such melting is often done using portable heating components which are attached to the contained in situ to form a heating arrangement for the container. The heating components used in such heating arrangements may include such components as electric heating jackets, base heaters, heat guns, and/or hot air blowers. This allows the temperature of the material in the container to be raised in a controlled fashion. Typically, the heating arrangement is applied until substantially all the material in the container has transitioned into the liquid phase, allowing for the material to be poured or otherwise extracted from the container. The heating arrangement is then usually dismantled from the container so that it may be used on further containers. In this way different locations may maintain inventories of heating components for heating arrangements suitable for the factors specific to that location, providing an advantage over heating arrangements that are integral to the containers.

The configuration of the heating arrangement required to quickly and efficiently raise the temperature of a material to melting point in a particular container depends on a large number of factors. These include, for example, the ambient temperature, the physical properties of the material in the container, the physical properties of the container itself, the power of the heating arrangement, and so on. Failure to select an appropriate heating arrangement can result in problems such as long times to establish a phase change of the material in the container and/or damage to the container through overheating. Raising the temperature of a material too quickly can also, in some case, cause damage or degradation to the material itself and even safety issues where rapid or over heating may result in the production of toxic fumes or a risk of explosion. As such, when any of the above factors change (for example when a new cargo is to be received at a new location) then it is necessary to determine suitable heating arrangements, providing appropriate time to reach a desired temperature or melting point.

Such heating arrangements are usually determined via trial and error by applying trial heating arrangements to containers filled with the material in question, often at the desired location. The material in the container is then monitored to determine how long the heating arrangement takes to melt the material. However, performing this heating task in real-time can be very time consuming (on the order of tens of hours, for example). This means there is very little overhead for trying out alternative configurations for the heating arrangement. Equally, it needs to be carried out with the cargo in situ often at the location where the heating arrangement is to be used so can be restrictive. In the cases where trial heating arrangements are too powerful, it can also result in damaged cargo and/or containers in the course of the tests. Equally, where a location has wide variation in ambient temperature it may not be possible to carry out such trials for the whole range of ambient temperature, this may mean that the most appropriate heating arrangement is ultimately not identified.

It is also important that the heating can be relied upon to completely melt the material as attempting to pump material with large amounts of solid remaining can be hazardous and damaging to the cargo and equipment.

These issues therefore, render the task of determining an appropriate heating arrangement difficult and subject to inaccuracy.

### SUMMARY OF THE INVENTION

It is therefore an object of the invention to provide systems and methods of determining a time to establish a phase change in a material disposed in a container arrangement, when subject to a pre-determined heating arrangement. In this way an optimized heating arrangement may be determined for a given combination of material and container arrangement.

In accordance with a first aspect, there is provided a method for calculating a time to establish a phase change of a material in a predetermined container arrangement subject to a heating (or temperature control) arrangement (such as one or more heat sources and/or one or more cooling elements). The method comprises the steps of: determining a set of basis functions from a plurality of temperature field snapshots for a corresponding material (which may be contained in a corresponding container arrangement and, optionally, subject to the heating arrangement); representing an interface between a solid region of the material and a liquid region of the material as a boundary surface; iteratively updating the boundary surface over time based on calculated (or determined) heat flux across the boundary to thereby update a volume of the solid region and a volume of the liquid region for the material in the container arrangement; and determining a time to establish a phase change of the material contained in the predetermined arrangement based on a convergence condition for the iterative updating. Each iteration comprises, calculating for the liquid region, using a temperature dependent thermal diffusivity function, a temperature distribution for the liquid region as a combination of the basis functions of the set of basis functions. Determining the set of basis functions may be done by (or may comprise) Proper Orthogonal Decomposition. The set of basis functions may be a reduced set of basis functions formed from a predetermined number of basis functions.

Each iteration may further comprise, calculating for the solid region, using a further thermal diffusivity function, a temperature distribution for the solid region as a combination of the basis functions.

In some examples of the above methods the calculation of the temperature distribution for the liquid and/or solid region may comprise solving the heat equation using the respective thermal diffusivity function. Additionally, or alternatively the calculating the temperature distribution may comprise determining, based on the set of basis functions and the respective thermal diffusivity function, a set of time-dependent coefficients corresponding to the set of basis functions.

Each of the set of finite elements may have a respective temperature, and the elements may be updated subject to a) an isothermal condition or b) a weak condition applied to the respective temperatures of the set of finite elements.

In some examples of the above methods the convergence condition may comprise a ratio of the volume of the solid region to the volume of the liquid region reaching a predetermined threshold value; or a proportion of the set of finite elements of the boundary surface reaching a predetermined location within the container arrangement.

In some examples of the above methods the temperature-dependent thermal diffusivity function for the liquid region may use the Boussinesq approximation. For example the temperature-dependent thermal diffusivity function for the liquid region, α, may be defined as:

$$α = \frac{k ( T )}{{C}_{p} ( T ) ( {p}_{l} - γ {ρ}_{l} ( T - {T}_{ref} ) )}$$

wherein T is an instantaneous temperature of the material, k(T) is a temperature-dependent thermal conductivity for the material, Cₚ(T) is a temperature-dependent specific heat capacity for the material, ρₗ is a fluid density of the material at a reference temperature T_{ref}, and γ is a linear thermal expansion factor for the material.

In accordance with a second aspect there is provided a method of selecting a heating arrangement for the liberation of a material from a container arrangement. The method comprises carrying out any of the methods set out above for each heating arrangement of a plurality of heating arrangements, to obtain a respective time to establish a phase change of the material for each heating arrangement; and selecting an optimal heating arrangement from the plurality based at least in part on the respective times to establish a phase change of the material for each heating arrangement.

In some example methods said step of selecting is based on a peak temperature of the material for each heating arrangement not exceeding a pre-determined threshold.

The selected heating arraignment may then be applied to the container arraignment to thereby liberate the material.

The invention also provides apparatus corresponding to, and comprising elements, modules or components arranged to put into effect the above methods, for example one or more various suitably configured computing devices.

In particular, the invention therefore provides a system (such as a phase change calculation module) for calculating a time to establish a phase change of a material in a predetermined container arrangement subject to a heating (or temperature control) arrangement (such as one or more heat sources and/or one or more cooling elements). The system comprises: a basis determining module arranged to determine a set of basis functions from a plurality of temperature field snapshots for a corresponding material (which may be contained in a corresponding container arrangement and, optionally, subject to the heating arrangement); a boundary surface module arranged to represent an interface between a solid region of the material and a liquid region of the material as a boundary surface; and to iteratively update the boundary surface over time based on calculated (or determined) heat flux across the boundary to thereby update a volume of the solid region and a volume of the liquid region for the material in the container arrangement, thereby determining a time to establish a phase change of the material contained in the predetermined arrangement based on a convergence condition for the iterative updating. Each iteration comprises, calculating for the liquid region, using a temperature dependent thermal diffusivity function, a temperature distribution for the liquid region as a combination of the basis functions of the set of basis functions.

The invention also provides a system for selecting a heating arrangement for the liberation of a material from a container arrangement. The system comprises: a phase change calculation module (such as the phase chance calculation module above) arranged to obtain a respective time to establish a phase change of the material for each heating arrangement of a plurality of heating arrangements; and a recommendation module arranged to select (or recommend) an optimal heating arrangement from the plurality based at least in part on the respective times to establish a phase change of the material for each heating arrangement.

The invention also provides one or more computer programs suitable for execution by one or more processors, such computer program(s) being arranged to put into effect the methods outlined above and described herein. The computer program may be stored on a computer-readable medium.

### BRIEF DESCRIPTION OF THE FIGURES

Embodiments of the invention will now be described, by way of example only, with reference to the accompanying drawings, in which:

FIG. 1 schematically illustrates a container arrangement (such as a bulk container) containing a pre-defined material;

FIG. 2a illustrates a phase change calculation system for determining (or calculating or otherwise identifying) a heating arrangement for use with a container arrangement of an end user;

FIG. 2b is a flow diagram schematically illustrating the process carried out by the phase change calculation system of FIG. 2a;

FIG. 3a illustrates a phase change calculation module in accordance with the phase change calculation system of FIG. 2a;

FIG. 3b is a flow diagram schematically illustrating an example method for calculating a time to establish a phase change in a material in a container arrangement subject to a heating arrangement, according to the phase change module of FIG. 3a;

FIG. 3c is a flow diagram schematically illustrating an example implementation of the step, the step of updating the boundary surface, of the method in FIG. 3b;

FIG. 4 schematically illustrates an example of a computer system which may be used to implement the systems as described in FIGS. 2a and 3a;

FIG. 5 is a flow diagram schematically illustrating an example implementation of the method for calculating a time to establish a phase change in a material in a container arrangement subject to a heating arrangement described in FIGS. 3a and 3b;

FIG. 6a illustrates an example container arrangement containing a paraffin wax material, subject to a heating arrangement composed of two 1000 W electric heating jackets;

FIG. 6b shows a graph of temperature against elapsed heating time for the container arrangement of FIG. 6a when subjected to the heating arrangement.

### DETAILED DESCRIPTION

In the description that follows and in the figures, certain embodiments of the invention are described. However, it will be appreciated that the invention is not limited to the embodiments that are described and that some embodiments may not include all of the features that are described below. It will be evident, however, that various modifications and changes may be made herein without departing from the scope of the invention as set forth in the appended claims.

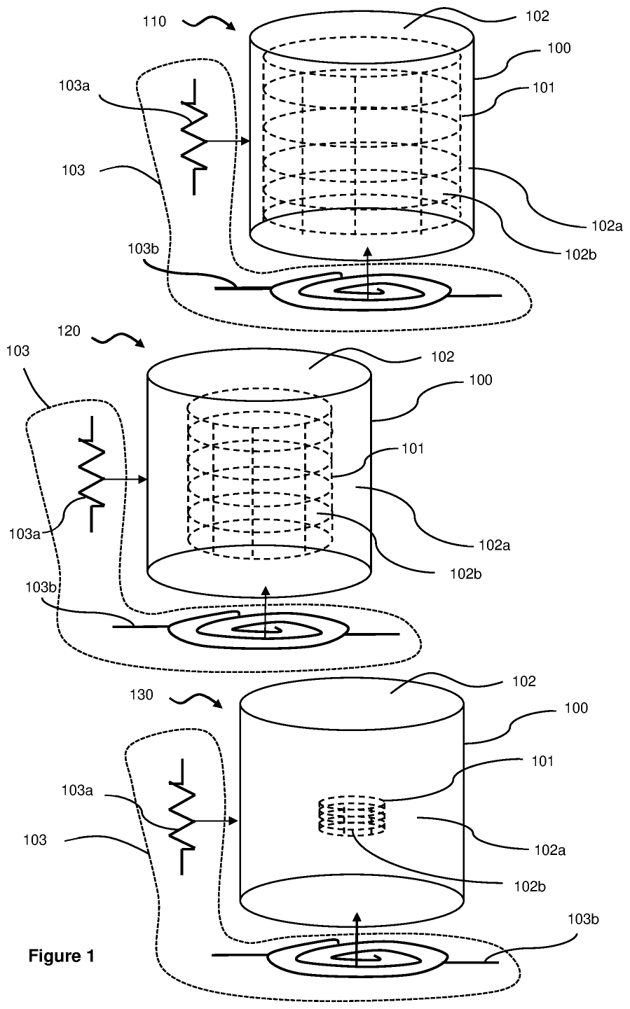

FIG. 1 schematically illustrates a container arrangement (such as a bulk container) 100 containing a pre-defined material 102. The container arrangement 100 shown is in the form of a drum for ease of understanding but it will be appreciated that any container arrangement 100 may be used. The container arrangement 100 is shown at three consecutive points in time 110; 120; 130. It will be appreciated that the FIG. 1 shows the time evolution of a container arrangement 100 subject to an external heating arrangement 103 as the material 102 contained within the container arrangement 100 melts under the influence of the heat supplied by the external heating arrangement 103. In particular, at an initial time 110 the material 102 is mostly in the solid phase, at a further time 120 an amount of the material 102 adjacent to the walls of the container arrangement 100 has melted and is in the liquid phase, and at a final time 130 substantially all of the material 102 is in the liquid phase. At this final time 130 the material is then capable of being discharged from the container arrangement 100.

In the example shown in FIG. 1, the external heating arrangement 103 comprises a first heating arrangement component 103a (which may be, for example, an external heating jacket surrounding an external side wall of the cylindrical container arrangement 100) and a second heating arrangement component 103b (which may be, for example, a base heater or hotplate located under the container arrangement 100). However it will be appreciated that the heating arrangement 103 may comprise any number of heating components 103a; 103b. A heating component may comprise (or be) one or more of an electrical component, a fluid component, or a gas component. For example, the heating component may comprise an electrical component capable of producing heat, such as a resistive coil or other electrical component that produces heat through work. In another example, the heating component may comprise a system of tubes or pipes for circulating a heated fluid or gas, and optionally a component for heating the fluid or gas. The heating component may be configured to regulate the amount of heat it produces, for example by including a temperature cut-off such as a temperature dependent switch or thermostat. The heating component may comprise one or more control systems, to control, for example, the temperature or power output of the heating component.

FIG. 1 further schematically illustrates an interface 101 moving through the material 102 held in the container arrangement 100 where the container arrangement 100 is subject to an external heating arrangement 103 comprising one or more heating components 103a, 103b. The interface 101 represents an interface between a liquid region (or liquid zone) 102a of the material 102 and a solid region (or solid zone) 102b of the material 102, and can be seen to move through the container arrangement 100 as heat from the heating arrangement 103 is continually applied to the container arrangement 100 over time.

In particular, for the first time 110 FIG. 1 shows the material 102 in the container arrangement 100 close to a time when the external heating arrangement 103 is first applied to the container arrangement 100 (e.g. within a few minutes from when the external heating arrangement is first applied to the container 100, depending on the material 102 in question). At this point, the solid region 102b occupies a large proportion of the container arrangement 100 relative to the liquid region 102a, as a temperature of an outer portion of the material 102 begins to rise subject to the heating arrangement 103.

For the further time 120, FIG. 1 then shows how the interface 101 has propagated through the material 102 under the influence of the external heating arrangement 103, sometime after the external heating arrangement 103 is first applied (e.g. approximately an hour from when the external heating arrangement 103 is first applied to the container 100, depending on the material 102 in question). At this point, the proportion of the solid region 102b relative to the liquid region 102a in the container arrangement 100 may be roughly equal.

Finally, for the final time 130, FIG. 1 shows how the interface 101 has propagated through the material 102 under the influence of the external heating arrangement 103, at a stage where nearly all of the material 102 has transitioned into the liquid phase (e.g. several hours from when the external heating arrangement 103 is first applied to the container 100, depending on the material 102 in question). At this point, the solid region 102b occupies a small proportion of the container arrangement 100 relative to the liquid region 102a, as the temperature throughout the material 102 starts to approach that of the external heating arrangement 103.

The external heating arrangement 103 may be continuously applied in order for a phase change to be established in the material 102. Alternatively, the external heating arrangement 103 may be intermittently applied for a phase change to be established in the material 102. In yet another alternative, the heating arrangement 103 may be an internal heating arrangement (not shown). In one example, the interface 101 could be a melting front. Alternatively, the interface 101 could be a solidification front, and the heating arrangement 103 could alternatively be a refrigeration arrangement.

In practice, the first time 110 shown in FIG. 1 illustrates a state of an end user's container arrangement 100 shortly after a heating arrangement 103 has been placed around the container arrangement 100 and has begun to apply heat to the container arrangement 100. The third time 130 shown in FIG. 1 then illustrates a state of an end user's container arrangement 100 close to, or at, the end of the heating process, where the heating arrangement 103 has transferred enough heat to the material 102 to melt a large proportion of the material 102 in the container arrangement 100. It is at this time, or shortly after, that the material 102 in the container arrangement 100 is in a suitable condition for liberation from the container arrangement 100.

It will be appreciated that a liberation (or discharge) condition for the material 102 may be that a certain proportion of the material 102 has transitioned into the liquid phase. Alternatively, the liberation condition may be that substantially all of the material 102 has transitioned into the liquid phase. In yet another alternative, the liberation condition may be that substantially all of the material 102 has transitioned into the liquid phase, and an average temperature of the liquid material is such that the viscosity of the liquid allows for pouring of the liquid from the container arrangement 100.

Having a reliable understanding of the time to establish the phase change of the material can enable the discharge or liberation of the material to start whilst the melting is still ongoing whilst reducing the risk of solid blockages occurring in the discharge equipment.

As suggested above, if heating by the heating arrangement 103 is too quick or vigorous, the container arrangement 100 and/or the material 102 in the container arrangement 100 could be damaged. Alternatively, if the heating arrangement 103 is underpowered or too small, the material in the container arrangement 100 may never reach the liberation condition, or could solidify again before the time comes to transfer the material 102 from the container arrangement 100. Determination (or calculation) of how long it will take a particular heating arrangement 103 to heat the material 102 to reach the liberation condition may therefore be advantageous to avoid damage to the container arrangement 100 and/or material 102, or to enable the material 102 to be liberated from the container arrangement 100. Additionally, or alternatively, determination (or calculation) of which particular heating arrangement 103 will be required to heat the material 102 to reach the liberation condition in a given time would also be advantageous. In light of these determinations, a recommendation can then be made as to which heating arrangement 103 is most appropriate for the required heating task.

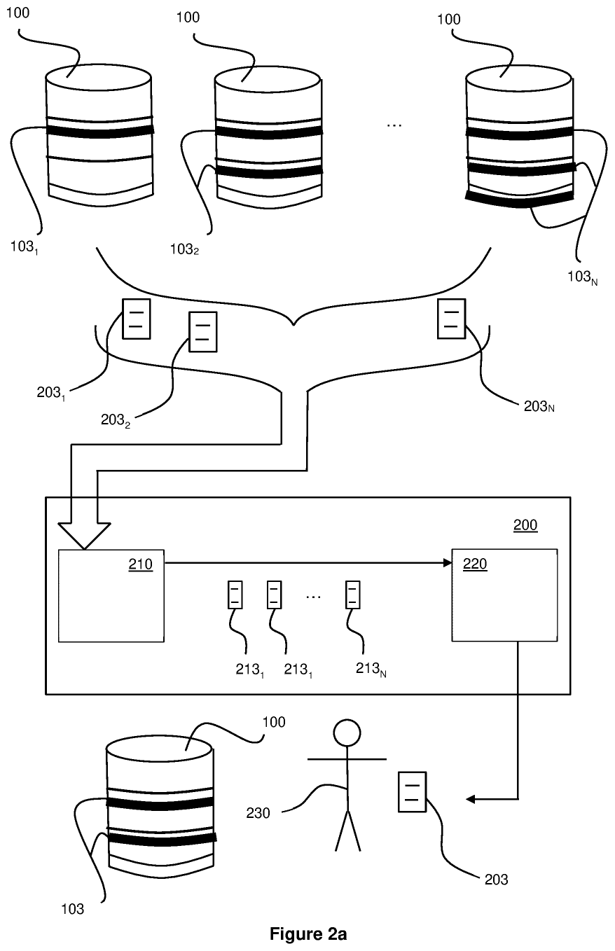

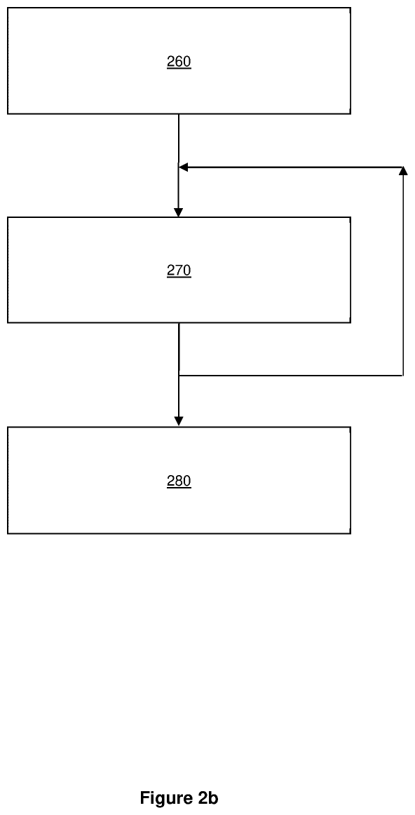

FIG. 2a illustrates a phase change calculation system 200 for determining (or calculating or otherwise identifying) a heating arrangement 103 for use with a container arrangement 100 of an end user 230. Reference numerals used in FIG. 2a in relation to the container arrangements 100, the material 102 and/or the solid and liquid regions 102a, 102b, and the heating arrangements 103 and/or heating arrangement components 103a, 103b represent the same or similar components as used with respect to FIG. 1.

As shown in FIG. 2a, there may be a plurality of different pre-defined heating arrangements 103₁, 103₂ . . . 103_{N} available for heating the container arrangement 100 of an end user 230. These pre-defined heating arrangements 103₁, 103₂ . . . 103_{N} may form or be part of a library of heating arrangements. Alternatively, or additionally, one or more of these pre-defined heating arrangements 103₁, 103₂ . . . 103_{N} may be created for use with the phase change calculation, for example based on end user requirements or specifications.

In one example, the container arrangement 100 may be heated using a first heating arrangement 103₁, which may comprise a single heating band or heating jacket (and/or other heating arrangement component(s)) placed around the container arrangement 100. In another example, the container arrangement 100 may be heated using a second heating arrangement 103₂, which may comprise a pair of heating bands or heating jackets placed around the container arrangement 100. In yet another example, the container arrangement 100 may be heated using an N^{th} heating arrangement 103_{N}, which may comprise a pair of heating bands or heating jackets placed around the container arrangement 100, in addition to a base heater or hotplate placed underneath the heating arrangement 100. While three examples have been given above, it will be appreciated that there may be any number of different heating arrangements 103 for heating the container arrangement 100 of the end user 203, and that these different heating arrangements 103 may each comprise any number of heating arrangement components.

The phase change calculation system 200 comprises a phase change calculation module 210 configured to calculate a transfer of heat from each of the plurality of heating arrangements 103₁, 103₂ . . . 103_{N} to the material 102 in the container arrangement 100 to thereby determine (or calculate or otherwise identify) a time to establish a phase change in the material 102 in the container arrangement 100 subject to each of the plurality of heating arrangements 103₁, 103₂ . . . 103_{N}. In other words, for each of the plurality of heating arrangements 103₁, 103₂ . . . 103_{N}, the phase change calculation module 210 is configured to calculate the movement of the interface 101 through the material 102 in the container arrangement 100 as heat from the respective heating arrangement 103 is applied to the container arrangement 100 over time. Thus, the phase change calculation module 210 is configured to determine the time it would take each of the plurality of heating arrangements 103₁, 103₂ . . . 103_{N} to heat the material 102 in the container arrangement 100 to establish a phase change in the material to allow it to be liberated from the container. The phase change calculation module 210 may provide or output the determined time to establish a phase change for each of the plurality of heating arrangements 103₁, 103₂ . . . 103_{N}, thereby providing a plurality of phase change times 213₁, 213₂ . . . 213_{N}.

In order to allow the phase change calculation module 210 to determine a time to establish a phase change in the material 102 in the container arrangement 100 for each of the plurality of heating arrangements 103₁, 103₂ . . . 103_{N}, a specification, schematic, or representation is obtained or created to represent the container arrangement 100 subject to each of the plurality of heating arrangements 103₁, 103₂ . . . 103_{N}. As will be discussed in more detail below, a representation of the container arrangement 100 subject to a respective heating arrangement 103 may comprise, for example, one or more of: a graphical representation (e.g. a CAD file, computer model, computer simulation, or schematic) of the container arrangement 100 (containing the material 102) subject to the respective heating arrangement 103; and a set of one or more properties of the container arrangement 100 and the respective heating arrangement 103. Each of the representations together form a set of representations 203₁, 203₂ . . . 203_{N}.

In one example, the set of representations 203₁, 203₂ . . . 203_{N} may be a plurality of data files 203₁, 203₂ . . . 203_{N} representing the container arrangement 100 subject to each of the plurality of heating arrangements 103₁, 103₂ . . . 103_{N}. For example: a first data file 203₁ may be obtained to represent the container arrangement 100 being subject to the first heating arrangement 103₁; a second data file 203₂ may be obtained to represent the container arrangement 100 being subject to the second heating arrangement 103₂; and an N^{th} data file 203_{N} may be obtained to represent the container arrangement 100 being subject to the N^{th} heating arrangement 103_{N}. Each of the plurality of data files 203₁, 203₂ . . . 203_{N} may be received by the phase change calculation module 210. It will be appreciated that the plurality of data files 203₁, 203₂ . . . 203_{N} may comprise one or more data files for the container arrangement 100 which are separate to one or more data files for the plurality of heating arrangements 103₁, 103₂ . . . 103_{N}.

In one example, the plurality of phase change times 213₁, 213₂ . . . 213_{N} may comprise: a first phase change time 213₁ corresponding to the time to establish a phase change in the material 102 in the container arrangement 100 subject to the first heating arrangement 103₁; a second phase change time 213₂ corresponding to the time to establish a phase change in the material 102 in the container arrangement 100 subject to the second heating arrangement 103₂; and an N^{th} phase change time 213_{N} corresponding to the time to establish a phase change in the material 102 in the container arrangement 100 subject to the N^{th} heating arrangement 103_{N}.

The plurality of phase change times 213₁, 213₂ . . . 213_{N} may be provided to a recommendation module 220 in order to allow a recommended heating arrangement to be provided to the end user 230, based on the determined times. For example, the recommendation module 220 may receive each of the plurality of phase change times 213₁, 213₂ . . . 213_{N} from the phase change calculation module 210. The recommendation module 220 may then compare the plurality of phase change times 213₁, 213₂ . . . 213_{N} to find the shortest time to establish a phase change in the material 102. The heating arrangement 103 corresponding to the shortest time to establish a phase change in the material 102 may then be recommended to the end user 230, which may be provided to the end user 230 as a specification, schematic, or representation 203 of the container 100 and recommended heating arrangement 103 as described above.

It will be understood that a variety of factors could be used in making the recommendation in addition to (or as an alternative to) the plurality of phase change times 213₁, 213₂ . . . 213_{N}. These factors may include, for example: a maximum temperature of each of the plurality of heating arrangements 103₁, 103₂ . . . 103_{N}, the container arrangement 100, and/or the material 102 (e.g. to determine whether the container arrangement 100 and/or material 102 may any suffer heat damage by one or more of the plurality of heating arrangements 103₁, 103₂ . . . 103_{N}); a peak, average, and/or total power consumption or output of each of the plurality of heating arrangements 103₁, 103₂ . . . 103_{N} (e.g. to determine the most energy intensive or energy efficient heating arrangement 103); and/or a cost of each of the plurality of heating arrangements 103₁, 103₂ . . . 103_{N}, such as a running cost, rental cost, or purchase cost (e.g. to determine the most cost-effective heating arrangement 103). The heating arrangement recommendation provided to the user may also be based one or more of these factors. For example, the shortest time to establish a phase change may result in an unacceptably large or high maximum temperature of the container arrangement 100 or material 102, so the shortest time to establish a phase change may need to be discarded in favour of the next shortest time to establish a phase change, which does not heat the container arrangement 100 and/or material 102 to an unacceptably high maximum temperature. The heating arrangement corresponding with said next shortest time would then be the recommended heating arrangement 103.

The resulting recommended heating arrangement may then be applied to the container arrangement to enable the liberation of the material form the container arrangement.

FIG. 2b is a flow diagram schematically illustrating a method 250 carried out by the phase change calculation system 200 of FIG. 2a.

At step 260, the phase change calculation system 200 receives a plurality of heating arrangements 103₁, 103₂ . . . 103_{N}. As described above, in one example, the plurality of heating arrangements 103₁, 103₂ . . . 103_{N} may be represented as a plurality of data files 203₁, 203₂ . . . 203_{N}, which may be received by the phase change calculation module 210 of the phase change calculation system 200.

At step 270, the phase change calculation system 200 determines a time to establish a phase change in the material in the container arrangement, subject to a respective one of the plurality of heating arrangements 103₁, 103₂ . . . 103_{N}. As will be described in more detail below, the phase change calculation module 210 of the phase change calculation system 200 calculates the movement of the interface 101 through the material 102 in the container arrangement 100 as heat from the respective heating arrangement is applied to the container arrangement 100 over time.

As can be seen in FIG. 2a, the step 270 of determining a time to establish a phase change in the material 102 in the container arrangement may be repeated. In particular, the step 270 of determining a time to establish a phase change in the material 102 in the container arrangement 100 may be repeated a number of times such that a time to establish a phase change in the material 102 is determined for each of the plurality of heating arrangements 103₁, 103₂ . . . 103_{N}. Thus, as described above, the phase change calculation module 210 may output a plurality of phase change times 213₁, 213₂ . . . 213_{N}, each of the plurality of phase change times 213₁, 213₂ . . . 213_{N} corresponding to a respective one of the plurality of heating arrangements 103₁, 103₂ . . . 103_{N}.

At step 280, a heating arrangement 103 for establishing a phase change in the material 102 in the container arrangement 100 is selected based on the determined times. For example, as described above, the recommendation module 220 of the phase change calculation system 200 may receive the plurality of phase change times 213₁, 213₂ . . . 213_{N} and provide a recommended heating arrangement 103 based on the plurality of phase change times 213₁, 213₂ . . . 213_{N}, and optionally other factors as set out above.

The recommended (or optimal) heating arrangement may then be applied to the container arrangement to thereby liberate the material.

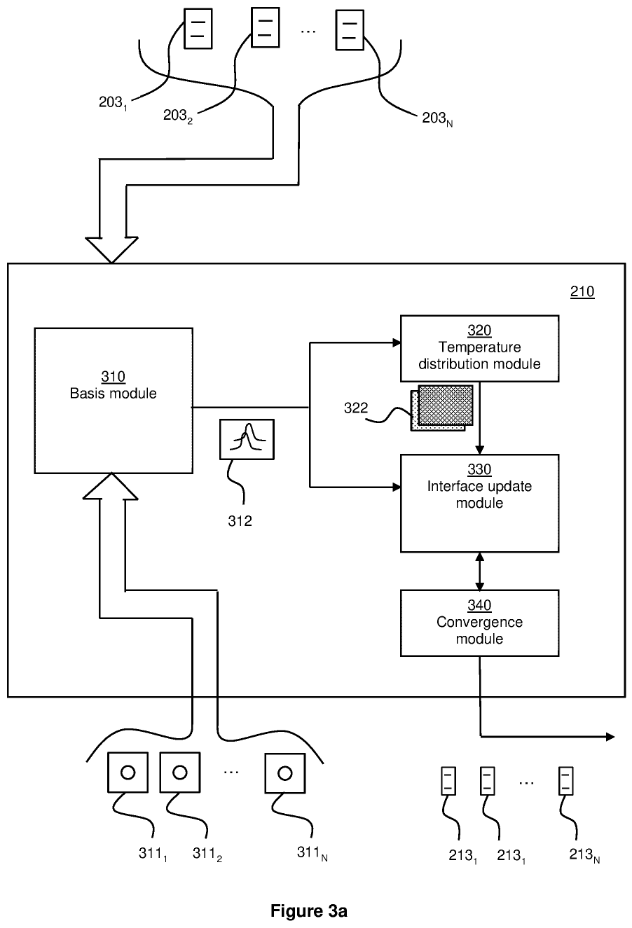

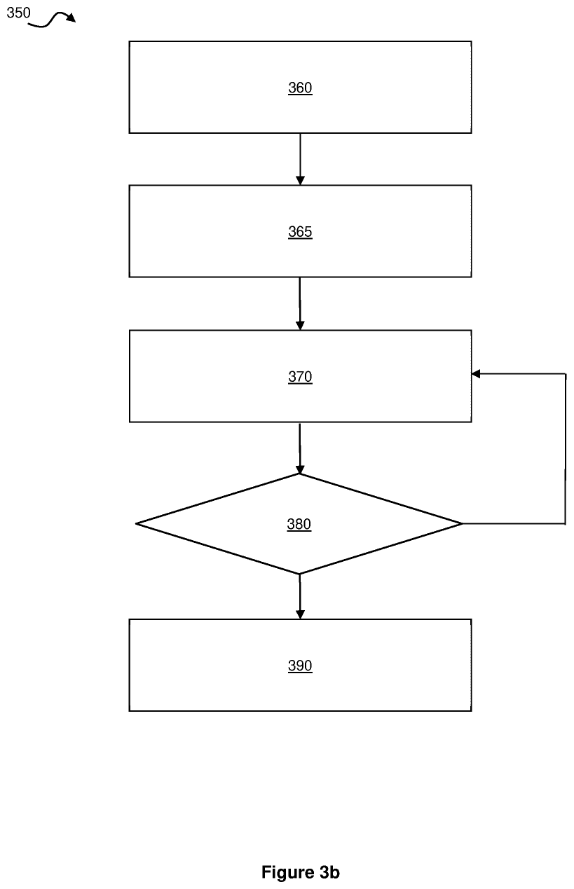

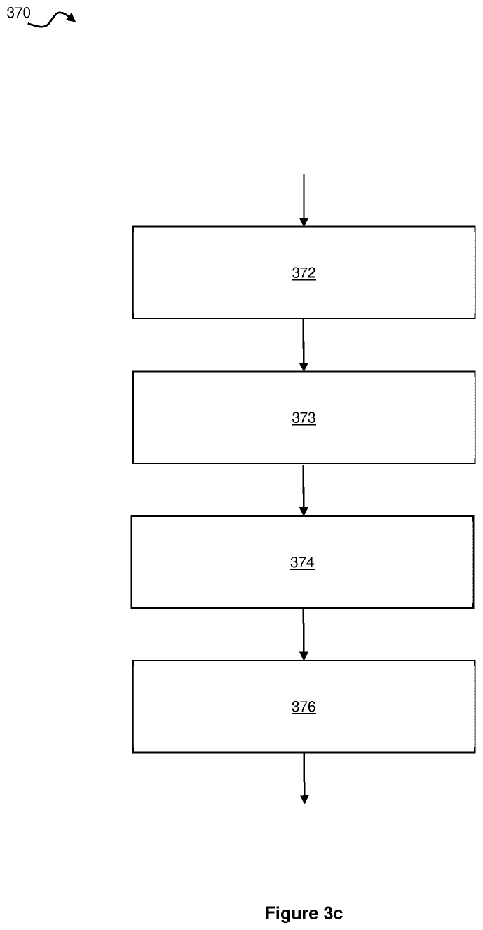

FIG. 3a illustrates a phase change calculation module 210 in accordance with the phase change calculation system 200 of FIG. 2a. Reference numerals used in FIG. 3a in relation to the container arrangements 100, the material 102 and/or the solid 102b and liquid regions 102a, the heating arrangements 103 and/or heating arrangement components 103a, 103b, the set of representations 203₁, 203₂ . . . 203_{N}, the plurality of phase change times 213₁, 213₂ . . . 213_{N}, and the phase change calculation module 210 represent the same or similar components as used with respect to FIGS. 1 and 2a.

As shown in 3a, the phase change calculation module 210 is configured to calculate a time to establish a phase change in a material 102 in a container arrangement 100 subject to a heating arrangement 103. In particular, as described in more detail, below the phase change calculation module 210 is arranged to iteratively update the interface or boundary surface 101 between the liquid and solid regions 102a, 102b of the material 102 over time based on a calculated heat flux across or through the boundary surface 101. The heat flux is calculated based on temperature distributions determined for the solid 102b and liquid 102a regions consistent with the regions being subject to the given container arrangement and heating arrangement. In particular the temperature distribution for the liquid region 102a is calculated by treating (or assuming or otherwise approximating) the liquid region 102a as a pseudo-solid region. In other words the temperature distribution for the liquid region 102a is determined using a suitable temperature dependent thermal diffusivity function as part of a reduced order model. The updating of the boundary surface causes the volume of each of the liquid and solid regions 102a, 102b for the material 102 in the container arrangement 100 to be updated. The updated volumes of the liquid and solid regions 102a, 102b may be used to check or test a convergence condition for the iteration and thus may be used determine whether or not the material 102 would be in a suitable condition for liberation from the container arrangement 100.

The phase change calculation module 210 may obtain or receive a representation 203 of the container arrangement 100 subject to heating arrangement 103 in the form of a data file or suitable specification as described above.

The phase change calculation module 210 comprises a basis determining module 310 configured to determine a set of basis functions 312 from a plurality of temperature field snapshots 3111, 3112 . . . 311N for a corresponding material. The plurality of temperature field snapshots 3111, 3112 . . . 311N describe (or cover or otherwise represent) an amount of the corresponding material heated from a solid state to a liquid state. In other words, the plurality of temperature field snapshots 3111, 3112 . . . 311N describe the phase change of a corresponding material from a solid to a liquid phase. The temperature field snapshots are typically generated (or calculated or otherwise obtained) from a numerical simulation of the phase change of the corresponding material. This simulation is carried out subject to an external heat source applied to the solid material. However, it will be appreciated that the temperature field snapshots may be instead be formed from experimental observations of heating an amount of the corresponding material.

The simulation used to generate the plurality of temperature snapshots is typically a full-order simulation (or a simulation using a full-order model). The full-order simulation may be run for sufficient time (or arranged such that) to observe a phase change from a solid phase to a liquid phase for all (or substantially all or at least part) of the corresponding material being simulated. Suitable full-order simulation techniques include any one of: the enthalpy-porosity model; the effective heat capacity method; the dynamic mesh with moving boundary technique; and so on. Whilst these full-order simulation techniques would be well known to the skilled person, for completeness a brief summary of some of these techniques is included below.
Enthalpy-porosity model: in this method the liquid-solid zone is treated as a porous media zone with porosity equal to liquid fraction. An example of this is described in Mohamed Fadl, P. C. E., 2019. Numerical investigation of the influence of mushy zone parameter Amush on heat transfer characteristics in vertically and horizontally oriented thermal energy storage systems. Applied Thermal Engineering, Issue 151, pp. 90-99.
Effective heat capacity method: in this method a pseudo-material with discontinuous temperature dependent material properties near the melting temperature simulates the melting front. An example of this is described in Nazzi Ehms, J. et al., 2019. Fixed Grid Numerical Models for Solidification and Melting of Phase Change Materials (PCMs) . . . . MDPI: Applied Sciences, Volume 49.
Dynamic mesh with moving boundary: in this method the solid zone and the liquid zone are two separate domains and the mesh interface between them will move according to the net heat flux on the boundary. An example of this is described in Martin, D. H. C. J. R. M. F. a. D. Z., 2016. Multiphase modelling of Melting/solidification with high density variation using XFEM, https://corpus.ulaval.ca/jspui/handle/20.500.11794/27140.
   It will be appreciated that implementations of these and other suitable full-order simulation methods may be found in well-known computational fluid dynamics software packages including ANSYS, COMSOL and OpenFOAM

The corresponding material in the snapshot calculation is described as a corresponding material insofar as it may have one or more properties which are similar to (or the same as) one or more properties of the material 102 in the container arrangement 100 for which a time to establish a phase change is to be calculated. In particular a material with a similar melting point and/or similar specific latent heat to the material 102 in the container arrangement 100 is preferred as the corresponding material. More preferably, the corresponding material may be required to have a melting point within 10 to 15% of the material 102 in the container arrangement 100 and/or a specific latent heat within 10 to 15% of the material 102 in the container arrangement 100. Through it will be appreciated that in some case the discrepancy between the material and the corresponding material may be higher without adversely impacting accuracy. Identification of a suitable corresponding material may be done using small scale test calculations as a matter of routine. It will be appreciated that the corresponding material may be the same as the material 102 in the container arrangement 100.

Each temperature snapshot therefore may be thought of as representing the temperature field within the corresponding material a respective point in time during the full-order simulation. In this way the temperature snapshots may describe the time evolution of the heating of the corresponding material. A temperature snapshot may comprise a scalar temperature field. The scalar temperature field represents the temperature distribution across the bulk corresponding solid material at a given point in time. As such, each temperature field snapshot typically comprises a set of temperature values for the corresponding material at a given point in time. Each temperature value corresponding to a respective spatial positon within the amount of corresponding material.

The basis functions are determined such that the temperature field snapshots can be reproduced by the sum of the products of the basis functions with a set of time dependent coefficients. In other words the basis functions are arranged to reproduce the spatially dependent behaviour of the temperature snapshots separately from the time dependent behaviour. Mathematically the basis functions may obey the relation:

$${T}_{t} ( x , y ) \approx {\sum }_{i = 1}^{M} {b}_{i , t} {ϕ}_{i} ( x , y )$$

where Tₜ(x, y) is the temperature snapshot for time t, ϕᵢ(x, y) is the i^{th} basis function (of which there are M in total) and bit is a coefficient for the i^{th} basis function at time t. It will be appreciated that the coefficient bit may equally be represented as a function of time bᵢ(t).

The basis functions may be determined by Principle Component Analysis (PCA) or Proper Orthogonal Decomposition (POD) techniques.

It will be appreciated that the basis functions may be thought of as encoding (or representing or otherwise accounting for) spatial correlations of temperature change for the material during the phase change process.

Once the set of basis functions 312 has been determined or obtained from the plurality of temperature field snapshots 3111, 3112 . . . 311N by the basis determining module 310, the set of basis functions 312 are made available to the temperature distribution module 320.

The determined set of basis functions 312 may allow for a reduction in the mode space or degrees of freedom of the phase change problem. In particular, the basis determining module 310 may be arranged to select (or generate) a subset of the basis functions available as the set of basis functions 312 for further use. Typically, the basis functions selected are the basis functions corresponding to highest eigenvalues (or energy modes). For example, the cumulative correlation energy Eₘ, may be used to determine the selection (e.g. truncation order) of the basis functions. After calculating the eigenvalues λᵢ for each basis function and sorting them in a descending order, the Eₘ for the first m out of n total basis functions is defined as:

$${E}_{m} = {\sum }_{i = 1}^{m} {λ}_{i} / {\sum }_{i = 1}^{n} {λ}_{i}$$

Preferably the number of basis function is chosen such that the value for Eₘ is 0.99 or higher. Typically, the number of basis functions required is around 10.

The temperature distribution module 320 is arranged to calculate for a given region a temperature distribution 322 for the region as a combination of the basis functions. For a liquid region 102a the temperature distribution module 320 is arranged to approximate the liquid as a solid with a temperature dependent thermal diffusivity.

In particular the temperature distribution module 320 is typically arranged to calculate the temperature distribution 322 by solving the heat equation. The heat equation may be expressed as:

$$\frac{\partial T ( t , x , y , z )}{\partial t} - α {∇}^{2} T ( t , x , y , z ) = 0$$

where T(t, x, y, z) is the temperature distribution 322 and a is the thermal diffusivity. As such, for the liquid region 102a the thermal diffusivity is a function of temperature. The thermal diffusivity function may be a function of the mean temperature of the region. Alternatively, the thermal diffusivity function may be a function of the temperature distribution 322. In this way the value of the thermal diffusivity function may vary with position in the region. As described shortly below the temperature distribution 322 is typically calculated as part of an iterative process. The thermal diffusivity function may therefore be dependent on a previous temperature distribution 322. Where a previous temperature distribution 322 is not available (such as for an initial iteration) the thermal diffusivity function may be dependent on an initial temperature distribution 322. The initial temperature distribution may be provided based on a starting temperature (or mean temperature, or predicted temperature) of the region. For example the initial temperature distribution may comprise a constant initial temperature across the region.

The temperature distribution 322 is calculated subject to the heating arrangement and the container arrangement 100. This is typically achieved by imposing a (or requiring or otherwise enforcing) a boundary condition on the temperature distribution 322 corresponding to the container arrangement 100 and the heating arrangement. For example the temperature distribution module 320 may require the temperature distribution 322 of the region at the interface with the container arrangement 100 to be a predetermined temperature (or temperatures) determined by the action of the heating arrangement. Additionally, or alternatively the temperature distribution module 320 may impose a predetermined heat flux to the region at the interface with the container arrangement 100 based on (or consistent with or determined by) the heat flux provided by the heating arrangement. The heat flux of the heating arrangement 103 may be derived, determined, or calculated from known properties of the heating arrangement 103, such as its power and/or surface area and/or arrangement in space (or relative to the container arrangement 100).

The temperature distribution 322 is represented by the temperature distribution module as a linear combination of the basis functions 312 scaled by respective coefficients. As such the temperature distribution 322 may be represented as:

$$T \approx {T}_{mean} ( x , y , z ) + {\sum }_{i}^{N} {b}_{i} ( t ) {ϕ}_{i} ( x , y , z )$$

where Tₘₑₐₙ is the mean temperature field over all of the snapshots, bᵢ(t) is a time dependent coefficient, ¢; (x, y, z) a basis function and N the number of basis functions in the set of basis functions 312. It will be appreciated that whilst the temperature distribution 322 may be represented using all of the basis functions determined from the temperature snapshots, N=M, a reduced set of basis functions 312 may be used instead, N<M. Typically the set of basis functions 312 is truncated so that only the most important basis functions are used to represent the temperature distribution 322. This is often done by using only the basis functions that correspond to the highest energy modes of the temperature snapshots (as discussed further below). This may be done by limiting to a predetermined number of basis functions, or limiting to basis functions having above a pre-determined threshold energy mode. Such thresholds may be determined by experimentation and/or testing for convergence with respect to the number of basis functions as discussed further below. The term Tₘₑₐₙ is included in the expression above for consistency with the mathematical formalism described later on herein. However, it will be appreciated that the time dependent coefficients may be selected to take account of this term. For example, the above expression could be written as

$$T \approx {\sum }_{i}^{N} {b}_{i}^{′} ( t ) {ϕ}_{i} ( x , y , z )$$

where bᵢ′(t)=bᵢ(t)+bᵢ⁽⁰⁾, and Σᵢ^{N}bᵢ⁽⁰⁾ϕᵢ(x, y, z)=Tₘₑₐₙ (x, y, z).

As such, the temperature distribution module 320 may be arranged solve a Galerkin projection of the heat equation on the set of basis functions 312 to obtain the temperature distribution 322 of a given region, at a given iteration. As set out shortly below it will be understood that known numerical methods (such as ordinary differential equation solvers and numerical integration) may be used to obtain the time dependant coefficients.

By using the basis functions from the temperature snapshots in solving the heat equation, the temperature distribution 322, may be determined with a significantly reduced computational effort relative to a full-order simulation. Additionally, the use of a solid approximation for the liquid region 102a, with a temperature dependent thermal diffusivity function allows the temperature distribution 322 for the liquid region 102a to be calculated without explicit calculation of any fluid dynamics effects occurring within the liquid region 102a, whilst maintaining a sufficient level of accuracy.

The phase change calculation module 210 comprises a boundary surface module 330. The boundary surface module 330 is arranged to represent (or calculate or generate) the interface between the solid region 102b and the liquid region 102a of the material 102 in the container arrangement 100 as a boundary surface. The boundary surface module 330 is also arranged to update the boundary surface based on a calculated heat flux across the boundary surface. The heat flux is calculated by the boundary surface module 330 based on temperature distributions of the solid and liquid regions provided by the temperature distribution module 320. It will be understood that the heat flux is proportional to the temperature gradient at the interface (or boundary surface). Typically, heat flux may be given as:

$$Q = - k ∇ T$$

where T is the temperature distribution 322 and k is the thermal conductivity of the material 102. The boundary surface module 330 may calculate the heat flux by calculating the heat flux for the solid region 102b and the heat flux for the liquid region 102a separately, where the overall (or net) heat flux is given by the difference. For example:

$$Q = ( - {k}_{s} ∇ {T}_{s} ) - ( - {k}_{l} ∇ {T}_{l} )$$

where Tₛ is the temperature distribution 322 of the solid region 102b, kₛ is the thermal conductivity of the solid material 102, T, is the temperature distribution of the liquid region 102a, k, is the thermal conductivity of the liquid material 102.

The boundary surface module 330 may be thought of as being arranged to update the boundary surface to the boundary surface after a given time interval (or time step) has elapsed. As described shortly below the boundary surface is typically updated as part of an iterative process. As such the time interval may be the time step of the iterative process.

The boundary surface module 330 may be arranged to calculate the rate of change (or velocity) of the boundary surface based on the calculated heat flux. More specifically the Stefan problem may be solved:

$$- ρ {L}_{f} \frac{{dx}^{*}}{dt} = - {k}_{s} ∇ {T}_{s} {\vert}_{{x}^{*}} + {k}_{l} ∇ {T}_{l} {\vert}_{{x}^{*}}$$

where ρ is the density of the material 102 (at the interface), Lf is the specific latent heat of fusion of the material 102 and x* is the boundary surface.

It will be appreciated that the boundary surface can be updated (or propagated) by a given time step using the calculated boundary surface velocity.

Typically, the boundary surface is represented as a set (or mesh) of finite elements (or cells or nodes). Each mesh cell (or node or element) may be explicitly tracked or updated based on the calculated velocity.

The Stefan problem above is solved numerically. The Stefan problem may be solved subject to a constraint such as enforcing a constant temperature across the boundary surface (for example requiring that each mesh cell has the same constant temperature). Preferably, however, a weak constraint is used. In particular requiring the local average of temperature at the boundary surface to be constant. In other words, the temperature of each of the set of finite elements of the boundary surface 101 may allowably deviate from an average temperature by a predetermined amount. Typically the average temperature (or the constant temperature) may be (or be related to) the melting point of the material 102. Both of these constraints are examples of an isothermal constraint, and in this way the boundary surface may be thought of as an isothermal surface. In this way the Stefan problem may be solved using standard numerical techniques involving Lagrangian multipliers. Examples of these are set out in Jonsson, T., 2013. On the one dimensional Stefan problem (Dissertation) http://urn.kb.se/resolve?urn=urn: nbn: se: umu: diva-80215 and are not discussed further herein.

It will be understood that the updating of the boundary surface based on the calculated heat flux set out above using finite elements is an example of a “moving boundary” method, also known as the Arbitrary Lagrangian-Eulerian method, for which any one of a number of known numerical solvers may be used including ANSYS, COMSOL, OpenFOAM, SIMSCALE and ABAQUS.

However, it will be appreciated that the boundary surface module 330 could make use of any number of numerical or analytical techniques to calculate the heat flux across the boundary surface 101 and move the boundary surface 101 within the material 102, based on the calculated temperature distributions for the solid and liquid regions.

The phase change calculation module 210 comprises a convergence module 340, configured to test or check whether a convergence condition for the movement of the boundary surface 101 has been satisfied or met. In a general sense, the convergence condition may be thought of as a check or test of how much of the material 102 in the container arrangement 100 has transitioned into the liquid phase. Put another way, the convergence condition may be thought of as a check or test of whether or not the material 102 would be in a suitable condition for liberation from the container arrangement 100.

In one example, the convergence condition may comprise a volume of at least one of the liquid region 102a of the material 102 and the solid region 102b of the material 102 reaching or exceeding (or falling below) a predetermined value. Taking a simple numerical example, for 1.00 cubic metre of material 102 in the container arrangement 100, the convergence condition may be that the volume of the liquid region 102a of the material 102 reaches or exceeds a value of 0.99 cubic metres. Taking another simple numerical example, for 1.00 cubic metre of material 102 in the container arrangement 100, the convergence condition may be that the volume of the solid region 102b of the material 102 reaches or falls below a value of 0.01 cubic metres. Of course, it will be appreciated that the predetermined values for the volumes of the liquid and/or solid regions 102a, 102b may be selected or predetermined based on the dimensions of the container arrangement 100 and/or material 102 being considered in the phase change calculation. It will also be appreciated that the volumes of the liquid and/or solid region 102a, 102b could be determined in a number of different ways. For example, a volume of the material 102 bounded by the boundary surface 101 may be compared with a total volume of the material 102 in the container arrangement 100.

In another example, the convergence condition may comprise a ratio of a volume of the liquid region 102a of the material 102 to a volume of the solid region 102b of the material 102 reaching or exceeding (or falling below) a predetermined value. Taking a simple numerical example, for 1.00 cubic metre of material 102 in the container arrangement 100, the convergence condition may be that the ratio of the volume of the liquid region 102a of the material 102 to the volume of the solid region 102b of the material 102 reaches or exceeds a value of 99 (e.g. equivalent to the ratio of the volume of the liquid region 102a to the volume of the solid region 102b where the liquid region 102a has a volume of 0.99 cubic metres and the solid region 102b has a volume of 0.01 cubic metres).

In another example, the convergence condition may comprise or one or more of the finite elements of the boundary surface 101 reaching a predetermined location in the material 102 and/or container arrangement 100. For example, it may be determined whether a coordinate of one or more finite elements of the boundary surface 101 has passed beyond a predetermined coordinate (or set of coordinates) of the representation of the material 102 and/or container arrangement 100.

As will be described in more detail below, if the convergence module 340 finds that the convergence condition is not reached, then the interface moving module 330 is configured to continue with the phase change calculation for the next time step. However, if the convergence module 340 finds that the convergence condition is reached, then the phase change calculation does not advance to the next time step. Instead, the phase change calculation module 210 is configured to determine the time to establish a phase change in the material 102 subject to the respective heating arrangement based on the convergence condition.

It will be understood that, as described above with respect to FIG. 2b, the phase change calculation module 210 is configured to repeat the process described above for each of the plurality of representations 203₁, 203₂ . . . 203_{N} to thereby determine a time to establish a phase change for each of the plurality of representations 203₁, 203₂ . . . 203_{N} (and therefore each of the plurality of heating arrangements 103₁, 103₂ . . . 103_{N}) and thus output or provide a plurality of phase change times 213₁, 213₂ . . . 213_{N}.

FIG. 3b is a flow diagram schematically illustrating an example method 350 for calculating a time to establish a phase change in a material 102 in a container arrangement 100 subject to a heating arrangement 103, according to the phase change module 210 of FIG. 3a.

At a step 360 a set of basis functions 312 is determined (or calculated) from a plurality of temperature field snapshots 3111, 3112 . . . 311N for a corresponding material. The step 360 may be carried out by the basis module 310 as described above. The temperature field snapshots are typically generated (or calculated or otherwise obtained) from a numerical simulation of the phase change of the corresponding material. The basis functions are determined such that the temperature field snapshots can be reproduced by the sum of the products of the basis functions with a set of time dependent coefficients.

At a step 365 the interface between a solid region 102b of the material 102 and a liquid region 102a of the material 102 is represented (or instantiated or defined or otherwise initialized) as a boundary surface. The boundary surface is defined at an initial time as an initial boundary surface corresponding to the solid and liquid regions in the container arrangement 100 at said initial time. It will be appreciated that at the initial time the material 102 may often be in a single initial state, such as solid or liquid. As such the initial boundary surface may correspond to the interface between the material 102 and the container arrangement 100. Alternatively, an initial non-zero volume of the other state may be enforced, typically between the region of the initial state and the container arrangement 100. Such a region of the other state is typically chosen to be of negligible volume, and simply aids natal convergence of the boundary surface.

Typically, the boundary surface is represented as a set (or mesh) of finite elements (or cells or nodes).

At a step 370 the boundary surface is updated for a pre-determined time step based on calculated heat flux across the boundary surface. In other words the boundary surface at a time t is updated to the boundary surface at time t+Δt, where Δt is the time step. This provides the boundary surface propagated forward in time in the heating (or cooling) process. As such the volume for the solid region 102b and a volume of the liquid region 102a for the material 102 in the container arrangement 100 is updated for the pre-determined time step. The step 370 comprises calculating for the liquid region 102a, using a temperature dependent thermal diffusivity function, a temperature distribution 322 for the liquid region 102a as a combination of the basis functions of the set of basis functions 312. As such the temperature distribution 322 for the liquid region 102a is calculated by simulating (or modelling) the liquid region 102a as a solid region (or pseudo solid region) with a temperature dependent thermal diffusivity.

At a step 380 a convergence condition for the boundary surface 101 is checked. The step 380 may be carried out by the convergence module 340. The convergence condition may typically comprise (or be related to) a pre-determined threshold of phase change having occurred (or being met or exceeded). This may be represented as any of: a ratio of the volumes of the solid and liquid regions, an absolute value of the volume of the solid and/or liquid region 102a, and so on. If the convergence condition is not met then the step 370 is iterated (or re-run) for the next time step. It will be appreciated that the iteration of the step 370 in effect results in the propagation of the calculation of the phase change in time by the pre-determined time steps. The time-steps (or intervals) may be of a constant length across the iterative process or may vary. It will be appreciated that the time-step (or time-steps) may be selected or adjusted based on convergence behaviour of the iterative process as is well-known in such iterative convergence methods. For example, the time step may be automatically varied during the iterative procedure based on the local and global Courant number and CFL conditions. As will be appreciated, Courant number determines the rate of simulation update based on the time step (Δt), element or mesh size (Δx) and the speed of the physical system (u).

$$C = u Δ t / Δ x$$

The CFL condition (if used) specifies that the maximum courant number must be less than 1 for most numerical methods.

If the convergence condition is met then at a step 390 the time to establish a phase change of the material 102 contained in the predetermined arrangement is determined. This is typically the sum of the time steps of the iterations of the step 370. In other words the total simulated (or simulation) time elapsed before the convergence condition was met. It will be appreciated that such a time is typically much longer than the actual real time required to carry out the method 350 allowing for many container and/or heating arrangements to be evaluated computationally much quicker than performing physical heating evaluations. The time to establish a phase change of the material 102 may be output to a user and/or used for further processing, as outlined above in the system 200 of FIG. 2a.

Additionally, by using the projected thermal diffusivity functions to update temperature distributions for each of the liquid region 102a and the solid region 102b, the phase change calculation can be focused upon the set of finite elements making up the boundary surface as opposed to a set of finite elements spread throughout both the solid and liquid regions. This avoids the need for carrying out complex meshing and/or re-meshing calculations during the phase change calculation, which are otherwise commonly associated with FEA. Put another way, by using the projected thermal diffusivity functions to update one or more temperature distributions for each of the liquid region 102a and the solid region 102b, the calculation progresses quickly to an examination of the finite elements of the boundary surface, without having to consider a set of finite elements spread throughout the material 102 at large.

FIG. 3c is a flow diagram schematically illustrating an example implementation of the step 370, the step of updating the boundary surface, of the method 350 in FIG. 3b.

At a step 372 a temperature distribution 322 is calculated for the liquid region 102a. The step 372 may be carried out by the temperature distribution module 320. The temperature distribution 322 for the liquid region is calculated as a combination of the basis functions, with the liquid approximated (or calculated as) as a solid (or pseudo solid) with a temperature dependent thermal diffusivity. Typically, as discussed below, the temperature dependent thermal diffusivity function is arranged to include (or take account of) the thermal expansion of the fluid. In particular, the temperature dependent thermal diffusivity may follow (or obey) the Boussinesq approximation. The temperature dependent thermal diffusivity function may be given by:

$$α ( T ) = \frac{k ( T )}{{C}_{p} ( T ) ( {ρ}_{l} - {γρ}_{l} ( T - {T}_{ref} ) )}$$

where k(T) is the thermal conductivity of the liquid material 102 (which may depend on temperature), Cₚ(T) is the specific heat capacity of the liquid material 102 (which may depend on temperature), γ is the linear thermal expansion factor of the liquid material 102, and ρₗ is the fluid density of the liquid material 102 at a temperature T_{ref}.

Typically the temperature used for the argument of the thermal diffusivity function is obtained from the temperature distribution 322 at the previous iteration (or time). As such, when calculating a temperature distribution 322 at t+Δt, i.e. T_{t+Δt}, the thermal diffusivity used is α(Tₜ). Where a previous temperature distribution 322 is not available (such as for an initial iteration) the thermal diffusivity function may be dependent on an initial temperature distribution. The initial temperature distribution may be provided based on a starting temperature (or mean temperature, or predicted temperature) of the region. For example the initial temperature distribution may comprise a constant initial temperature across the region, for example consistent with the container arrangement 100 and the material 102 being at thermal equilibrium with the ambient temperature.

As described above, the temperature distribution 322 is calculated subject to the heating arrangement and the container arrangement 100. This is typically achieved by imposing a (or requiring or otherwise enforcing) a boundary condition on the temperature distribution 322 corresponding to the container arrangement 100 and the heating arrangement.

The temperature distribution 322 is represented as a linear combination of the basis functions 312 scaled by respective coefficients. As such the temperature distribution 322 may be represented as:

$$T \approx {T}_{mean} ( x , y , z ) + {\sum }_{i}^{N} {b}_{i} ( t ) {ϕ}_{i} ( x , y , z )$$

where Tₘₑₐₙ is the mean temperature field over all of the snapshots, bᵢ(t) is a time dependent coefficient, ϕᵢ(x, y, z) a basis function and N the number of basis functions in the set of basis functions 312. As set out shortly below it will be understood that known numerical methods (such as ordinary differential equation solvers and numerical integration) may be used to obtain the time dependant coefficients.

At a step 373 a temperature distribution 322 is calculated for the solid region 102b. The step 373 may be carried out by the temperature distribution module 320. The temperature distribution 322 for the solid region is calculated as a combination of the basis functions. The temperature distribution 322 for the solid region 102b may be calculated as set out above for the liquid region 102a. The temperature dependent thermal diffusivity for the solid region 102b may take the form:

$$α ( T ) = \frac{k ( T )}{{C}_{p} ( T ) {ρ}_{s}}$$

where k(T) is the thermal conductivity of the solid material 102 (which may depend on temperature), Cₚ(T) is the specific heat capacity of the solid material 102 (which may depend on temperature) and ρₛ is the density of the solid material 102. In some cases the thermal diffusivity of the solid region 102b may be assumed to be non-temperature dependent.

As described above, the temperature distribution 322 is calculated subject to the heating arrangement and the container arrangement 100. This is typically achieved by imposing a (or requiring or otherwise enforcing) a boundary condition on the temperature distribution 322 corresponding to the container arrangement 100 and the heating arrangement.

At a step 374 the net heat flux across the boundary surface is calculated. The step 374 may be carried out by the boundary surface module 330. The net heat flux across the boundary surface is calculated based on the temperature distributions of the solid and liquid regions. The step 374 may comprised calculating the net heat flux by calculating the heat flux for the solid region 102b and the heat flux for the liquid region 102a separately, where the overall (or net) heat flux is given by the difference. For example:

$$Q = ( - {k}_{s} ∇ {T}_{s} ) - ( - {k}_{l} ∇ {T}_{l} )$$

where Tₛ is the temperature distribution 322 of the solid region 102b, kₛ is the thermal conductivity of the solid material 102, T, is the temperature distribution 322 of the liquid region 102a, k, is the thermal conductivity of the liquid material 102.

At a step 376 the boundary surface is updated for a pre-determined time step based on the calculated heat flux across the boundary surface. The step 376 may comprise calculating the rate of change (or velocity) of the boundary surface based on the calculated heat flux. More specifically the Stefan problem may be solved:

$$- ρ {L}_{f} \frac{{dx}^{*}}{dt} = - {k}_{s} ∇ {T}_{s} {\vert}_{{x}^{*}} + {k}_{l} ∇ {T}_{l} {\vert}_{{x}^{*}}$$

where ρ is the density of the material 102 (at the interface), Lf is the specific latent heat of fusion of the material 102 and x* is the boundary surface.

It will be appreciated that the boundary surface can be updated (or propagated) by a given time step using the calculated boundary surface velocity. Typically, the boundary surface is represented as a set (or mesh) of finite elements (or cells or nodes). Each mesh cell (or node or element) may be explicitly tracked or updated based on the calculated velocity. The step 376 may comprise using a “moving boundary” method (or Arbitrary Lagrangian-Eulerian method) for updating the positions of the finite elements of the boundary surface.

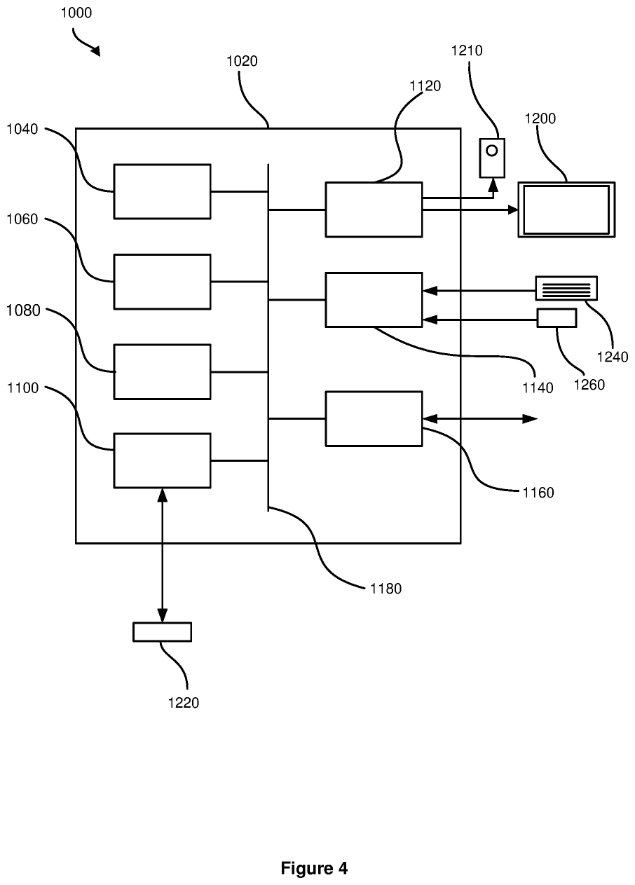

FIG. 4 schematically illustrates an example of a computer system 1000 which may be used to implement the systems 200 and 250 as described herein before. The system 1000 comprises a computer 1020. The computer 1020 comprises: a storage medium 1040, a memory 1060, a processor 1080, an interface 1100, a user output interface 1120, a user input interface 1140 and a network interface 1160, which are all linked together over one or more communication buses 1180.

The storage medium 1040 may be any form of non-volatile data storage device such as one or more of a hard disk drive, a magnetic disc, an optical disc, a ROM, etc. The storage medium 1040 may store an operating system for the processor 1080 to execute in order for the computer 1020 to function. The storage medium 1040 may also store one or more computer programs (or software or instructions or code).

The memory 1060 may be any random access memory (storage unit or volatile storage medium) suitable for storing data and/or computer programs (or software or instructions or code).

The processor 1080 may be any data processing unit suitable for executing one or more computer programs (such as those stored on the storage medium 1040 and/or in the memory 1060), some of which may be computer programs according to embodiments of the invention or computer programs that, when executed by the processor 1080, cause the processor 1080 to carry out a method according to an embodiment of the invention and configure the system 1000 to be a system according to an embodiment of the invention. The processor 1080 may comprise a single data processing unit or multiple data processing units operating in parallel or in cooperation with each other. The processor 1080, in carrying out data processing operations for embodiments of the invention, may store data to and/or read data from the storage medium 1040 and/or the memory 1060.

The interface 1100 may be any unit for providing an interface to a device 1220 external to, or removable from, the computer 1020. The device 1220 may be a data storage device, for example, one or more of an optical disc, a magnetic disc, a solid-state-storage device, etc. The device 1220 may have processing capabilities—for example, the device may be a smart card. The interface 1100 may therefore access data from, or provide data to, or interface with, the device 1220 in accordance with one or more commands that it receives from the processor 1080.

The user input interface 1140 is arranged to receive input from a user, or operator, of the system 1000. The user may provide this input via one or more input devices of the system 1000, such as a mouse (or other pointing device) 1260 and/or a keyboard 1240, that are connected to, or in communication with, the user input interface 1140. However, it will be appreciated that the user may provide input to the computer 1020 via one or more additional or alternative input devices (such as a touch screen). The computer 1020 may store the input received from the input devices via the user input interface 1140 in the memory 1060 for the processor 1080 to subsequently access and process, or may pass it straight to the processor 1080, so that the processor 1080 can respond to the user input accordingly.

The user output interface 1120 is arranged to provide a graphical/visual and/or audio output to a user, or operator, of the system 1000. As such, the processor 1080 may be arranged to instruct the user output interface 1120 to form an image/video signal representing a desired graphical output, and to provide this signal to a monitor (or screen or display unit) 1200 of the system 1000 that is connected to the user output interface 1120. Additionally or alternatively, the processor 1080 may be arranged to instruct the user output interface 1120 to form an audio signal representing a desired audio output, and to provide this signal to one or more speakers 1210 of the system 1000 that is connected to the user output interface 1120.

Finally, the network interface 1160 provides functionality for the computer 1020 to download data from and/or upload data to one or more data communication networks.

It will be appreciated that the architecture of the system 1000 illustrated in FIG. 4 and described above is merely exemplary and that other computer systems 1000 with different architectures (for example with fewer components than shown in FIG. 4 or with additional and/or alternative components than shown in FIG. 4) may be used in embodiments of the invention. As examples, the computer system 1000 could comprise one or more of: a personal computer; a server computer; a mobile telephone; a tablet; a laptop; a television set; a set top box; a games console; other mobile devices or consumer electronics devices; etc.

### Further Mathematical Detail

As set out above, the basis functions for the temperature distribution 322 can be calculated using proper orthogonal decomposition techniques from the temperature snapshots. To aid understanding an example is presented below.

Here the temperature snapshots are be arranged into a matrix. The eigenvectors of the temperature snapshot are then generated. The basis functions may then be generated as the sum of the product of the eigenvectors for each the temperature snapshot scaled by the respective temperature snapshot. In other words a temperature snapshot matrix T may be constructed according to:

$$T = ( \begin{matrix}{T}_{1 , 1} & {T}_{1 , 2} & … & {T}_{1 , M} \\ {T}_{2 , 1} & {T}_{2 , 2} & … & {T}_{2 , M} \\ ⋮ & ⋮ & ⋱ & ⋮ \\ {T}_{P , 1} & {T}_{P , 2} & … & {T}_{P , M}\end{matrix} )$$

where T_{k,j} indicates the j^{th} entry of the k^{th} temperature snapshot, where there are P snapshots each comprising M temperature values such that a given temperature snapshot Tₖ={T_{k,1}, T_{k,2}, . . . , T_{k,N}}. It will be appreciated that a temperature snapshot being a scalar field over space may be represented in a discretized form as a list of temperature values at particular points in space.

The eigenvalue problem to be solved may then be:

$$\frac{1}{N} ( T - {T}_{mean} ) \qquad {( T - {T}_{mean} )}^{T} β = βΛ$$

where Tₘₑₐₙ is a matrix of the mean temperatures over all of the snapshots

$${T}_{mean} = ( \begin{matrix}{T}_{mean}^{( 1 )} & … & {T}_{mean}^{( 1 )} \\ ⋮ & ⋱ & ⋮ \\ {T}_{mean}^{( N )} & … & {T}_{mean}^{( N )}\end{matrix} ) where {T}_{mean}^{( j )} = \frac{1}{P} {\sum }_{k = 1}^{P} {T}_{k , j} ,$$

and Λ is the diagonal matrix of eignenvalues

$$Λ = ( \begin{matrix}{λ}_{1} \\ ⋮ \\ {λ}_{N}\end{matrix} ) I .$$

Thus the basis functions may be generated as:

$${ϕ}_{i} ( x , y ) = {\sum }_{k = 1}^{P} {β}_{k} ( {T}_{k , i} ( x , y ) - {T}_{mean}^{( k )} ( x , y ) ) \qquad i = 1 , 2 , 3 , … , M$$

Where βₖ is the k^{th} column vector of the matrix β (i.e. the k^{th} eigenvector). It will be appreciated that for any given set of temperature snapshots these basis functions may be calculated numerically using Eigenvalue solving techniques or Single Value Decomposition solving techniques. Examples of suitable software packages include the Eigenvalue study step in the COMSOL Multiphysics software available from COMSOL Inc. Burlington MA, Unites States of America.

Again, as set out above, the heat equation is typically solved using a Galerkin projection on the basis functions, at least for the liquid region 102a, to obtain the temperature distribution 322.

Continuing the above example, the heat equation

$$\frac{\partial T}{\partial t} - α {∇}^{2} T = 0$$

may be projected onto the basis function space by taking the inner product of the basis functions with the temperature distribution 322:

$${\int }_{x , y , z} {ϕ}_{j} ( x , y , z ) . ( \frac{\partial T}{\partial t} - α {∇}^{2} T ) dxdydz = 0$$

Substituting the basis function representation of the temperature distribution 322, T=Σᵢ^{N}bᵢ(t)ϕᵢ(x, y, z), into this projection yields the following system of linear differential equations:

$${A}_{i , j} \frac{{db}_{i} ( t )}{dt} + {B}_{i , j} {b}_{i} ( t ) + {C}_{i} = 0 \qquad i , j = 1 , 2 , 3 , … , N$$

Where the coefficients A, B and C are defined as:

$${A}_{i , j} = {\int }_{x , y , z} {ϕ}_{j} . {ϕ}_{i} dxdydz = {δ}_{ij} = \{ \begin{matrix}0 & i \neq j \\ 1 & i = j\end{matrix} \\ {B}_{i , j} = {\int }_{x , y , z} {αϕ}_{j} ( \frac{{\partial }^{2} {ϕ}_{i}}{\partial {x}^{2}} + \frac{{\partial }^{2} {ϕ}_{i}}{\partial {y}^{2}} + \frac{{\partial }^{2} {ϕ}_{i}}{\partial {z}^{2}} ) dxdydz \\ {C}_{i} = {\int }_{x , y , z} {αϕ}_{j} ( \frac{{\partial }^{2} {T}_{mean}}{\partial {x}^{2}} + \frac{{\partial }^{2} {T}_{mean}}{\partial {y}^{2}} + \frac{{\partial }^{2} {T}_{mean}}{\partial {z}^{2}} ) dxdydz$$

The derivation of the coefficients A, B, and C can be seen from the following which, for simplicity show only the x, y dimensions. Defining the heat equation as of ∂T/∂t−α∇²T=0, to apply the Galerkin method consider an arbitrary test function v (x, y). Multiplying v (x, y) to both sides and integrating over the special domain results the following equation.

$${\int }_{x , y} \frac{\partial T}{\partial t} v ( x , y ) dxdy = {\int }_{x , y} α \frac{{\partial }^{2} T}{\partial {x}^{2}} v ( x , y ) dxdy + {\int }_{x , y} α \frac{{\partial }^{2} T}{\partial {y}^{2}} v ( x , y ) dxdy$$

The right-hand side integrals can be simplified using the integration by parts method.

$${{\int }_{x , y} α \frac{{\partial }^{2} T}{\partial {x}^{2}} v ( x , y ) = - ( α \frac{\partial T}{\partial x} v ( x , y ) ) \vert}_{\begin{matrix}x , y \\ boundary\end{matrix}} + {\int }_{x , y} α \frac{\partial T}{\partial x} \frac{\partial v ( x , y )}{\partial x} dxdy$$

Considering a homogenous boundary conditions the boundary term is equal to zero and the final weak form of the heat equation can be written as follows.

$${\int }_{x , y} \frac{\partial T}{\partial t} v ( x , y ) dxdy = {\int }_{x , y} α [ \frac{\partial T}{\partial x} \frac{\partial v ( x , y )}{\partial x} + \frac{\partial T}{\partial y} \frac{\partial v ( x , y )}{\partial y} ] dxdy$$

Assuming there exists a solution for T in the special domain, an approximate solution can be written using the POD method.

$$T ( x , y , t ) \approx {T}_{mean} ( x , y ) + {\sum }_{i}^{N} {b}_{i} ( t ) {ϕ}_{i} ( x , y )$$

Substituting the approximate solution with the test function v (x, y)=ϕⱼ(x, y) into the weak form of the heat equation results the following equation.

$${\int }_{x , y} {\sum }_{i = 1}^{N} \frac{{db}_{i} ( t )}{dt} {ϕ}_{i} ( x , y ) {ϕ}_{j} ( x , y ) dxdy = {\int }_{x , y} α [ {\sum }_{i = 1}^{N} {b}_{i} ( t ) \frac{\partial {ϕ}_{i} ( x , y )}{\partial x} \frac{\partial {ϕ}_{j} ( x , y )}{\partial x} + \frac{\partial {T}_{mean}}{\partial x} \frac{\partial {ϕ}_{j} ( x , y )}{\partial x} + {\sum }_{i = 1}^{N} {b}_{i} ( t ) \frac{\partial {ϕ}_{i} ( x , y )}{\partial y} \frac{\partial {ϕ}_{j} ( x , y )}{\partial y} + \frac{\partial {T}_{mean}}{\partial y} \frac{\partial {ϕ}_{j} ( x , y )}{\partial y} ] dxdy$$

Which can be rearranged into a first-degree ODE.

$${A}_{i , j} \frac{{db}_{i} ( t )}{dt} + {B}_{i , j} {b}_{i} ( t ) + {C}_{i} = 0 \qquad i , j = 1 , 2 , 3 , … , N$$

Where the coefficients A, B and C are defined as:

$${A}_{i , j} = {\int }_{x , y , z} {ϕ}_{i} . {ϕ}_{j} dxdy = {δ}_{ij} = \{ \begin{matrix}0 & i \neq j \\ 1 & i = j\end{matrix} \\ {B}_{i , j} = {\int }_{x , y , z} α ( \frac{\partial {ϕ}_{i} ( x , y )}{\partial x} \frac{\partial {ϕ}_{j} ( x , y )}{\partial x} + \frac{\partial {ϕ}_{i} ( x , y )}{\partial y} \frac{\partial {ϕ}_{j} ( x , y )}{\partial y} ) dxdy \\ {C}_{i} = {\int }_{x , y , z} α ( \frac{\partial {T}_{mean}}{\partial x} \frac{\partial {ϕ}_{j} ( x , y )}{\partial x} + \frac{\partial {T}_{mean}}{\partial y} \frac{\partial {ϕ}_{j} ( x , y )}{\partial y} ) dxdy$$

As set out previously, for at least the liquid region 102a, the thermal diffusivity, α, is temperature dependent and so may take as its argument the temperature distribution 322 T. In order to maintain the linearity of the above equations the temperature distribution 322 for the previous time step, that has already been calculated, is used instead as the argument to the thermal diffusivity function.

As such, the coefficients A, B, and C may be calculated by standard numerical integration. The coefficients bᵢ(t) may then be calculated by solving the system of linear differential equations. This may be done using numerical methods such as the Ordinary Differential Equation solver in the COMSOL Multiphysics software package.

In the example set out above a homogenous Neumann boundary condition has been used for temperature distribution 322 assuming a constant heat flux around the entire boundary of the domain. It will be appreciated that this simplifies the above calculation of the temperature distribution 322. Additionally, it has the benefit of rendering the calculating of the temperature distribution 322 more stable with respect to differences between the material 102 in the container arrangement 100 and the corresponding material used for generating the temperature snapshots. The temperature distribution 322 is then corrected to take account of the non-homogenous heat flux provided by the heating apparatus and the adjacent other region of the material 102. This can be done using standard numerical heat transfer solvers, such as the heat transfer solver available in the COMSOL Multiphysics software package, as discussed in COMSOL Reference Manual 5.5 U.S. Plant Pat. No. 1,182 and 1183, https://doc.comsol.com/5.5/doc/com.comsol.help.comsol/COMSOL ReferenceMan ual.pdf, which is incorporated herein by reference.

The resulting temperature distribution 322 arsing form the corrected coefficients may then be used as described previously to calculate the net heat flux across the boundary surface and then update said boundary surface for the given iteration. The temperature distribution 322 can then also be used as the input to the temperature dependent thermal diffusivity function for the region in the subsequent iteration.

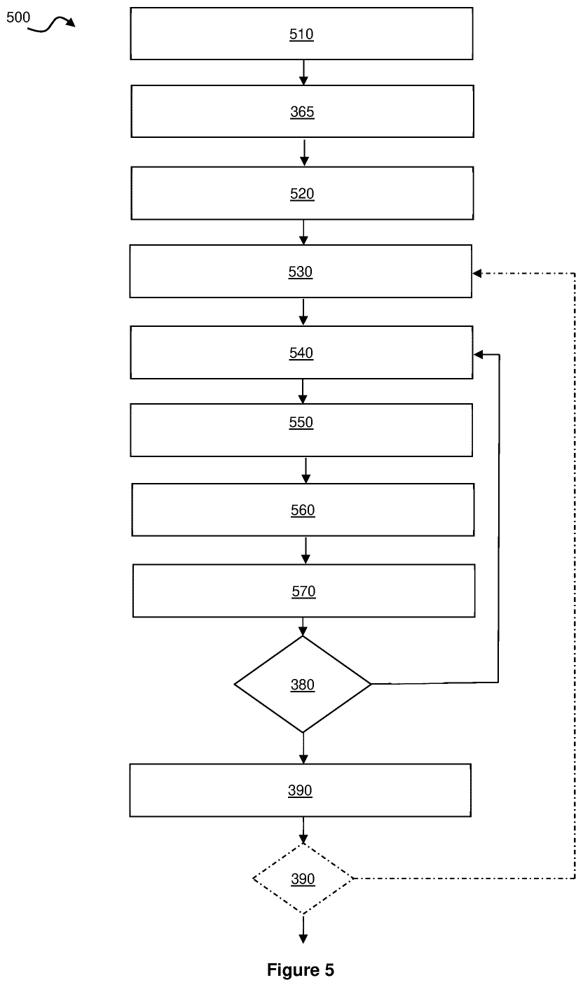

FIG. 5 is a flow diagram schematically illustrating an example implementation of the method 350 for calculating a time to establish a phase change in a material 102 in a container arrangement 100 subject to a heating arrangement 103.

At a step 510 a plurality of temperature field snapshots 3111, 3112 . . . 311N for a corresponding material are obtained, as described previously.

At a step 365 the interface between a solid region 102b of the material 102 and a liquid region 102a of the material 102 at an initial time is represented (or instantiated or defined or otherwise initialized) as a boundary surface, as described previously above.

At a step 520 an initial temperature distribution for the solid region 102b and an initial temperature distribution for the liquid region 102a are determined for an initial time. As set out previously the initial temperature distributions may be based on an ambient temperature of the environment in which the container is to be situated. For example the initial temperature distribution for the solid region 102b may be assumed to be a constant temperature at (or around) the ambient temperature. The initial temperature for the liquid region 102a may be assumed to be at or around the melting point of the material 102. Typically, to aid in numerical stability the initial temperature of the liquid region is set 2-3 degrees Celsius above the melting point of the material 102. In this way abnormal mesh velocities at the start of the calculation may be avoided.

At a step 530 a set of basis functions 312 is determined (or calculated) from the plurality of temperature field snapshots 3111, 3112 . . . 311N for the corresponding material. In particular, the basis functions are determined using POD techniques, in line with the relation:

$${ϕ}_{i} ( x , y ) = {\sum }_{k = 1}^{P} {β}_{k} {T}_{k , i} ( x , y ) \qquad i = 1 , 2 , 3 , … , M$$

described previously. The basis functions in the set of basis functions 312 are limited to a pre-determined number of the basis functions with the highest eigenvalues.

At a step 540 the A, B, and C coefficients outlined above are calculated for each of the solid and liquid regions at the current time. For at least the liquid region 102a the A, B, and C coefficients are calculated using a temperature dependent thermal diffusivity function, a (T), taking as its argument the temperature distribution 322 for the liquid region 102a. As set out previously, the A, B, and C coefficients in this example are calculated numerically using numerical integration methods as would be well understood by the skilled person.

At a step 550 a set of time dependent coefficients, b; (t), are calculated for each region by solving the systems of linear differential equations specified by the coefficients A, B, and C. The time dependent coefficients are determined subject to homogenous Neumann boundary conditions assuming a constant heat flux around each of the solid and liquid regions. Typically, this heat flux is assumed to be zero, as the corrected (or true) boundary conditions may be applied as part of step 560 described below. Respective temperature distributions for the solid and liquid region 102a are formed (or determined) as superpositions (or linear combinations) of the basis functions of the set of basis functions 312 scaled by the respective set of determined time dependent coefficients.

At a step 560 the temperature distributions for each region are adjusted for the boundary conditions resulting from the heat flux into each region caused by the heating arrangement and the other region. As set out above this is done using standard numerical heat transfer solvers, such as the heat transfer solver available in the COMSOL Multiphysics software package. These adjust the time dependent coefficents accordingly. It will be understood that the resulting temperature distributions for the solid and liquid region 102a are the temperature distributions for the current time in the iterative process.

At a step 570 the net heat flux across the boundary surface is calculated, as described previously above. The current time is updated by a pre-determined time step (as discussed previously) and the boundary surface is updated according to this new current time. As set out previously in the discussion of the step 376 of FIG. 3c this step includes calculating the mesh velocity for each finite element of the boundary surface according to the calculated net heat flux. The position of each element is then advanced (or updated) for the new current time based on the respective mesh velocity.

At a step 380 a convergence condition for the boundary surface 101 is checked, as described previously. If the convergence condition is not met then the method 500 returns to the step 540 the new current time.

If the convergence condition is met then at a step 390 the time to establish a phase change of the material 102 contained in the predetermined arrangement is determined. The time to establish a phase change of the material 102 may be output to a user and/or used for further processing, as outlined above in the system 200 of FIG. 2a.

It will be appreciated that at a further optional step 590 the convergence of the time to establish a phase change with respect to the number of basis functions used may determined. In this way the method may be iterated from the step 520 changing the number of basis functions and the convergence of the time to establish the phase change may be tested (or observed or determined). As such an optimum number of basis functions may be determined based on the minimum number of basis functions required for the time to establish a phase change to be converged to with a pre-determined threshold (or accuracy). Typically, as set out previously the basis functions selected are those with the highest eigenvalues (or energy modes) in this way such iteration may be seen as iteratively including further basis functions in order of descending energy mode. Equally, the iterative process may be arranged to iteratively reduce the number of basis functions in order of increasing energy mode.

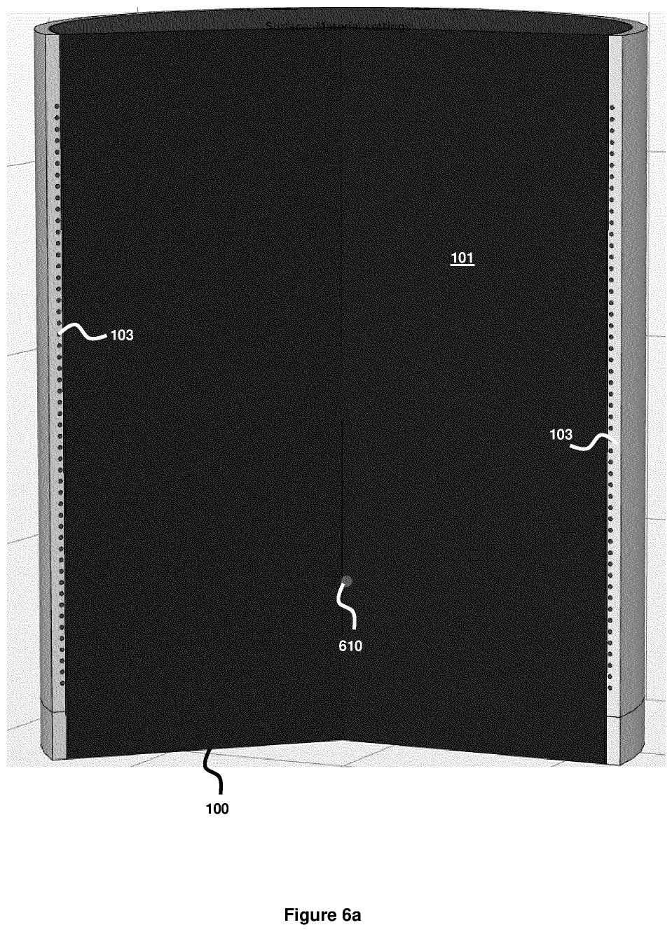

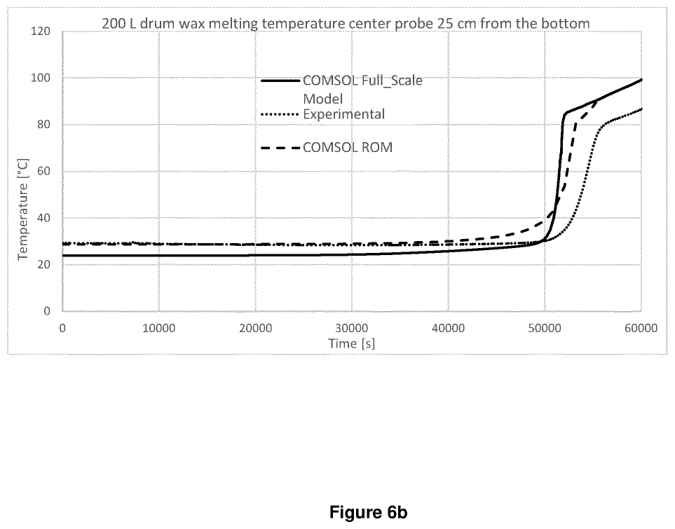

FIG. 6a illustrates an example container arrangement 100 containing a paraffin wax material 102, subject to a heating arrangement 103 composed of two 1000 W electric heating jackets. The container arrangement 100 is a standard 200 L drum. The paraffin wax material 102 has the following properties (as set out in Agus Dwi Korawan, S. S. W. W. D. W., 2017. 3D Numerical and Experimental Study on Paraffin Wax Melting in Thermal Storage for the Nozzle-and-Shell, Tube-and-Shell, and Reducer-and-Shell Models. Modelling and Simulation in Engineering, Volume 2017, pp. 1687-5591):

| Density | 750/(0.001 (T − | |
|---|---|---|
| | 319.15) + 1) kg/m³ | |
| Specific heat | 3100 | J/kgK |
| Thermal conductivity (liquid) | 0.21 | W/mK |
| Thermal conductivity (solid) | 0.12 | W/mK |
| Viscosity | 0.001 exp(−4.25 + | |
| | 1790/T) Ns/m² | |
| Latent heat | 166000 | J/kg |

| | |
|---|---|
| Solidus temperature | 321.7K |
| Liquidus temperature | 328.6K |

Also shown is a location 610 of a temperature probe.

FIG. 6b shows a graph of temperature (measured at the location 610 of the temperature probe) against elapsed heating time for the container arrangement 100 of FIG. 6a when subjected to the heating arrangement 103.

The dotted line shows the experimental results when the actual container arrangement was subject to the heating arrangement. The solid line shows the temperature as determined using a full order calculation using COMSOL as described above. The dashed line shows the temperature as determined using the methods of the invention set out above, i.e. the reduced order calculations. Here COMSOL was used to perform the calculations required for the method of the invention set out above.

As can be seen the method of the invention tracks the experimentally determined temperature curve closely. The phase transition times for the measured points are detailed at the following table. The time is measured between the solidus temperature and liquids temperature.

| | Transition start | Transition end | Duration |
|---|---|---|---|
| Experimental | 53639[s] | 54098[s] | 459[s] |
| Full-scale model | 50978[s] | 51405[s] | 427[s] |
| ROM | 51225[s] | 51663[s] | 438[s] |

The total heating time elapsed for the heating experiment was 16 hours and 40 minutes. By contrast the required calculation time (i.e. the real time needed to calculate the same elapsed heating time) was 8 hours and 20 minutes for the full order calculation and only 1 hour and 13 minutes for the method of the invention.

It will be appreciated that the methods described have been shown as individual steps carried out in a specific order. However, the skilled person will appreciate that these steps may be combined or carried out in a different order whilst still achieving the desired result.

It will be appreciated that embodiments of the invention may be implemented using a variety of different information processing systems. In particular, although the figures and the discussion thereof provide an exemplary computing system and methods, these are presented merely to provide a useful reference in discussing various aspects of the invention. Embodiments of the invention may be carried out on any suitable data processing device, such as a personal computer, laptop, personal digital assistant, mobile telephone, set top box, television, server computer, etc. Of course, the description of the systems and methods has been simplified for purposes of discussion, and they are just one of many different types of system and method that may be used for embodiments of the invention. It will be appreciated that the boundaries between logic blocks are merely illustrative and that alternative embodiments may merge logic blocks or elements, or may impose an alternate decomposition of functionality upon various logic blocks or elements.

It will be appreciated that the above-mentioned functionality may be implemented as one or more corresponding modules as hardware and/or software. For example, the above-mentioned functionality may be implemented as one or more software components for execution by a processor of the system. Alternatively, the above-mentioned functionality may be implemented as hardware, such as on one or more field-programmable-gate-arrays (FPGAs), and/or one or more application-specific-integrated-circuits (ASICs), and/or one or more digital-signal-processors (DSPs), and/or other hardware arrangements. Method steps implemented in flowcharts contained herein, or as described above, may each be implemented by corresponding respective modules; multiple method steps implemented in flowcharts contained herein, or as described above, may be implemented together by a single module.

It will be appreciated that, insofar as embodiments of the invention are implemented by a computer program, then a storage medium and a transmission medium carrying the computer program form aspects of the invention. The computer program may have one or more program instructions, or program code, which, when executed by a computer carries out an embodiment of the invention. The term “program” as used herein, may be a sequence of instructions designed for execution on a computer system, and may include a subroutine, a function, a procedure, a module, an object method, an object implementation, an executable application, an applet, a servlet, source code, object code, a shared library, a dynamic linked library, and/or other sequences of instructions designed for execution on a computer system. The storage medium may be a magnetic disc (such as a hard drive or a floppy disc), an optical disc (such as a CD-ROM, a DVD-ROM or a BluRay disc), or a memory (such as a ROM, a RAM, EEPROM, EPROM, Flash memory or a portable/removable memory device), etc. The transmission medium may be a communications signal, a data broadcast, a communications link between two or more computers, etc.

1. A method for calculating a time to establish a phase change of a material in a predetermined container arrangement subject to a heating arrangement, the method comprising the steps of:
determining a set of basis functions from a plurality of temperature field snapshots for a corresponding material;
representing an interface between a solid region of the material and a liquid region of the material as a boundary surface;
iteratively updating the boundary surface over time based on calculated heat flux across the boundary to thereby update a volume of the solid region and a volume of the liquid region for the material in the container arrangement,
each iteration comprising, calculating for the liquid region, using a temperature dependent thermal diffusivity function, a temperature distribution for the liquid region as a combination of the basis functions of the set of basis functions; and
determining a time to establish a phase change of the material contained in the predetermined arrangement based on a convergence condition for the iterative updating.

2. The method of claim 1, wherein each iteration comprises, calculating for the solid region, using a further thermal diffusivity function, a temperature distribution for the solid region as a combination of the basis functions.

3. The method of claim 1, wherein calculating the temperature distribution for the region comprises solving the heat equation using the respective thermal diffusivity function.

4. The method of claim 1, wherein calculating the temperature distribution for the region comprises:
determining, based on the set of basis functions and the respective thermal diffusivity function, a set of time-dependent coefficients corresponding to the set of basis functions.

5. The method of claim 1, wherein determining the set of basis functions comprises Proper Orthogonal Decomposition.

6. The method of claim 1 wherein the set of basis functions is a reduced set of basis functions formed from a predetermined number of basis functions.

7. The method of claim 1, wherein each of the set of finite elements has a respective temperature, and wherein the elements are updated subject to a) an isothermal condition or b) a weak condition applied to the respective temperatures of the set of finite elements.

8. The method of claim 1, wherein the convergence condition comprises either:
a ratio of the volume of the solid region to the volume of the liquid region reaching a predetermined threshold value; or
a proportion of the set of finite elements of the boundary surface reaching a predetermined location within the container arrangement.

9. The method of claim 1, wherein the corresponding material is contained in a corresponding container arrangement.

10. The method of claim 9, wherein the corresponding material is subject to the heating arrangement.

11. The method of claim 1, wherein the temperature-dependent thermal diffusivity function for the liquid region uses the Boussinesq approximation.

12. The method of claim 11, wherein the temperature-dependent thermal diffusivity function for the liquid region, a, is defined as:
α=k(T)/Cₚ(T)(ρₗ−γρₗ(T−T_{ref}))
wherein T is an instantaneous temperature of the material, k(T) is a temperature-dependent thermal conductivity for the material, Cₚ(T) is a temperature-dependent specific heat capacity for the material, ρₗ is a fluid density of the material at a reference temperature T_{ref}, and γ is a linear thermal expansion factor for the material.

13. The method of claim 1, wherein the heating arrangement is one of a plurality of heating arrangements, and the method further comprises,
for each of the other heating arrangements of the plurality of heating arrangements:
determining a set of basis functions from a plurality of temperature field snapshots for a corresponding material;
representing an interface between a solid region of the material and a liquid region of the material as a boundary surface;
iteratively updating the boundary surface over time based on calculated heat flux across the boundary to thereby update a volume of the solid region and a volume of the liquid region for the material in the container arrangement, each iteration comprising, calculating for the liquid region, using a temperature dependent thermal diffusivity function, a temperature distribution for the liquid region as a combination of the basis functions of the set of basis functions; and
determining a time to establish a phase change of the material contained in the predetermined arrangement based on a convergence condition for the iterative updating;
wherein the method further comprises selecting an optimal heating arrangement from the plurality of heating arrangements based at least in part on the respective times to establish a phase change of the material for each heating arrangement.

14. The method of claim 13 wherein said step of selecting is based on a peak temperature of the material for each heating arrangement not exceeding a pre-determined threshold.

15. The method of claim 13 further comprising applying the selected heating arrangement to the container arrangement to thereby liberate the material.

16. An information processing system comprising one or more processors arranged to carry out the following steps for calculating a time to establish a phase change of a material in a predetermined container arrangement subject to a heating arrangement:
determine a set of basis functions from a plurality of temperature field snapshots for a corresponding material;
represent an interface between a solid region of the material and a liquid region of the material as a boundary surface;
iteratively update the boundary surface over time based on calculated heat flux across the boundary to thereby update a volume of the solid region and a volume of the liquid region for the material in the container arrangement, each iteration comprising, calculating for the liquid region, using a temperature dependent thermal diffusivity function, a temperature distribution for the liquid region as a combination of the basis functions of the set of basis functions; and
determine a time to establish a phase change of the material contained in the predetermined arrangement based on a convergence condition for the iterative updating.

17. A non-transitory computer-readable medium storing instructions which, when executed by one or more processors of an information processing system, cause the information processing system to carry out a method for calculating a time to establish a phase change of a material in a predetermined container arrangement subject to a heating arrangement, the method comprising the steps of:
determining a set of basis functions from a plurality of temperature field snapshots for a corresponding material;
representing an interface between a solid region of the material and a liquid region of the material as a boundary surface;
iteratively updating the boundary surface over time based on calculated heat flux across the boundary to thereby update a volume of the solid region and a volume of the liquid region for the material in the container arrangement, each iteration comprising, calculating for the liquid region, using a temperature dependent thermal diffusivity function, a temperature distribution for the liquid region as a combination of the basis functions of the set of basis functions; and
determining a time to establish a phase change of the material contained in the predetermined arrangement based on a convergence condition for the iterative updating.

18. (canceled)
